(19) United States
(12) Patent Application Publication
(10) Pub. No.: US 2026/0364225 A1
(43) Pub. Date: Nov. 19, 2026

(54) GELATIN SPONGE MICROSPHERE PRODUCT AND PREPARATION METHOD THEREOF

(71) Applicant: Sichuan Dachuan Heyi Biotechnology Co., Ltd., Chengdu City (CN)

(72) Inventors: Chuan ZHANG, Chengdu City (CN); Xiangyuan QI, Chengdu City (CN); Quanwei CAI, Chengdu City (CN); Xin REN, Chengdu City (CN)

(21) Appl. No.: 19/659,051

(22) Filed: Apr. 27, 2026

(30) Foreign Application Priority Data
May 9, 2025 (CN) 202510593902.9

(51) Int. Cl. A61L 24/10; A61L 24/00

(52) U.S. Cl. CPC A61L 24/104; A61L 24/0036; A61L 2400/04

(57) ABSTRACT

A gelatin sponge microsphere product and its preparation method are provided. In the present disclosure, gelatin is dissolved in deionized water to obtain a dispersed phase solution; vegetable oil and a surfactant are mixed to obtain a continuous phase solution; vegetable oil and a surfactant are mixed to obtain a receiving phase solution; the dispersed phase solution and the continuous phase solution are injected respectively into a microfluidic device to obtain gelatin droplets; the gelatin droplets are introduced into the receiving phase solution, cooled, and solidified to obtain a crude product; the crude product is added to a first crosslinking solution for preliminary crosslinking fixation, and after removing the upper layer solution, a second crosslinking solution is added for further crosslinking fixation to obtain a semi-finished microsphere product; the upper layer solution of the semi-finished microsphere product is removed, and after washing, and the product is obtained.

### CROSS REFERENCE TO RELATED APPLICATION

This application claims priority to and benefits of Chinse patent application No. 202510593902.9, filed on May 9, 2025 (“Chinese Priority Application”). The entire contents of the Chinese Priority Application are incorporated herein by reference.

### TECHNICAL FIELD

The present disclosure relates to the technical field of materials, and in particular to a gelatin sponge microsphere product and preparation method thereof.

### BACKGROUND

Knee osteoarthritis (KOA) is one of the most common chronic pain diseases, with an extremely high incidence, especially among middle-aged and elderly people. Currently, early treatment for patients with KOA mainly includes oral medication, physical therapy, intra-articular injection of hyaluronic acid, etc. However, the effects are often not significant. Patients with severe or late-stage KOA need surgical treatment, mainly including arthroscopic debridement and knee replacement surgery. In 2015, Okuno, et al. first reported the use of transcatheter genicular artery embolization (GAE) to treat and relieve knee pain in patients with mild and moderate KOA, achieving good clinical results. Genicular artery embolization is a minimally invasive intra-arterial interventional method with advantages such as minimal trauma, high precision, and rapid onset of action, which can effectively relieve knee pain.

Hemoptysis refers to a clinical emergency of hemoptysis with a daily bleeding volume exceeding 500 mL or a per-episode bleeding volume exceeding 200 mL. Due to the complexity of the causes of hemoptysis, medical treatment is often ineffective for some patients. In 1974, French scholar Remy first reported the successful application of selective bronchial artery embolization (BAE) to treat massive hemoptysis. BAE is a minimally invasive interventional treatment method that selectively injects embolic material into a specific bronchial artery through a catheter, blocking blood flow to the corresponding vessel and thus controlling bleeding. This technology has the following significant advantages: precise control, accurately locating the lesion area through imaging guidance, reducing damage to healthy tissue; minimally invasive treatment, requiring only a rice-grain-sized puncture in the groin area, resulting in rapid postoperative recovery; treatment of various lung problems, not only treating hemostasis, but also being useful for recurrent bleeding in lung cancer and inhibiting tumor growth.

Lower gastrointestinal bleeding (LGIB) accounts for approximately 20% of acute gastrointestinal bleeding events, with an estimated incidence of 33-87/100,000, defined as bleeding originating from the distal end of the duodenojejunal junction (including the small intestine, colon, rectum, and anus). Although LGIB tends to resolve spontaneously and has milder clinical presentation than upper gastrointestinal bleeding (UGIB), mortality is closely related to comorbidities, ranging from 3% to 18%. Current guidelines recommend emergency CT angiography to locate the bleeding site in patients with hemodynamic instability and suspected persistent LGIB, followed by endoscopic or interventional treatment. Selective transarterial embolization (TAE) has become an effective and well-tolerated treatment option for patients with LGIB, and current literature emphasizes its safety.

However, the embolization microspheres currently used to treat the above clinical symptoms via interventional methods are mostly non-degradable materials, meaning that these microspheres need to remain permanently in the patient's body. Foreign body residue always leads to some complications, and ectopic embolization of microspheres can also occur during the embolization process. Gelatin is an animal protein composed of various amino acids, which has excellent water absorption and plasticity, good tissue compatibility, is easy to obtain, and is biodegradable in vivo. Embolization microspheres prepared from gelatin are a new type of peripheral vascular embolization agents developed in recent years.

In the existing embolization microsphere preparation methods, most of them first use freezing technology to pre-freeze the microspheres, then remove the oil solution by washing, then crosslink the microspheres using a crosslinking solution, and finally remove the crosslinking agent by washing the microspheres. The main technical shortcomings of the method of freezing-washing-crosslinking-washing are that: gelatin microspheres easily swell and become larger in the crosslinking solution, and the resulting microspheres are often larger in size, which is not conducive to the preparation of small particle size microspheres.

### SUMMARY

In view of this, the technical problem to be solved by the present disclosure is to provide a method for preparing gelatin sponge microspheres. The gelatin sponge microspheres prepared by the method of the present disclosure have a surface, which is smooth, wrinkle-free and uniformly crosslinked.

The present disclosure provides a method for preparing gelatin sponge microspheres, including the following steps:
A) dissolving gelatin in deionized water to obtain a dispersed phase solution; mixing vegetable oil and a surfactant to obtain a continuous phase solution; mixing vegetable oil and a surfactant to obtain a receiving phase solution;
B) injecting the dispersed phase solution and the continuous phase solution respectively into a microfluidic device to obtain gelatin droplets;
C) introducing the gelatin droplets into the receiving phase solution, cooling, and solidifying to obtain crude product of gelatin sponge microspheres;
D) adding the crude product of gelatin sponge microspheres to the first crosslinking solution for preliminary crosslinking; after removing the upper layer solution, adding the second crosslinking solution for further crosslinking to obtain semi-finished gelatin sponge microspheres;
E) removing the upper layer solution from the semi-finished gelatin sponge microspheres, and washing to obtain the gelatin sponge microsphere product.

In some specific embodiments, the temperature for dissolving gelatin in step A) is 35-70 °C, and the gel strength of gelatin is 100-300 bloom; the mass fraction of gelatin in the dispersed phase solution is 1%-50%; the stirring time is 0.5-2 h.

In some specific embodiments, the vegetable oil in step A) includes one or more of soybean oil, peanut oil, and corn oil; the surfactant is one or more of Span 80, Span 85, and PGPR; the surfactant mass fraction is 0.5% to 10%;

In the continuous phase solution, the mass ratio of the vegetable oil to the surfactant is 100:(1-10);

In the receiving phase solution, the mass ratio of the vegetable oil to the surfactant is 100:(0.1-2).

In some specific embodiments, the pressure of the dispersed phase in step B) is 0.1 kPa-500 kPa, and the pressure of the continuous phase is 0.05 MPa-1 MPa.

In some specific embodiments, the first crosslinking solution in step D) includes water, a crosslinking agent, and an organic solvent, wherein the crosslinking agent is an aldehyde organic compound; the organic solvent is one of n-octanol, n-butanol, propylene glycol, isopropanol, or acetone; the volume fraction of the crosslinking agent in the first crosslinking solution is 0.1%-1%, and the volume concentration of the organic solvent is 25%-50%; the time of the preliminary crosslinking is 10 min-60 min

In some specific embodiments, the second crosslinking solution in step D) includes water, a crosslinking agent, and an organic solvent, wherein the crosslinking agent is an aldehyde organic compound; the organic solvent is one of n-octanol, n-butanol, propylene glycol, isopropanol, or acetone; the volume fraction of the crosslinking agent in the second crosslinking solution is 0.1%-10%, and the volume concentration of the organic solvent is 25%-50%; the time of the further crosslinking is 10 min-2 h.

In some specific embodiments, the cooling temperature in step C) is preferably <30 °C; more preferably 0-8 °C. In some specific embodiments, the washing in step E) is washing with a cleaning agent followed by washing with deionized water; the cleaning agent is one of n-octanol, n-butanol, propylene glycol, isopropanol, or acetone; the volume concentration of the cleaning agent solution is not less than 75%.

In some specific embodiments, the microfluidic device in step B) includes a constant temperature environment chamber, a microfluidic constant pressure pump, and a microfluidic chip, wherein the microfluidic chip is a multi-channel microfluidic chip with a flow focusing structure; the multi-channel microfluidic chip of the present disclosure is preferably a 72-channel microfluidic chip.

The multi-channel microfluidic chip includes an upper cover plate containing an inlet and an outlet, an intermediate functional plate containing microchannels, and a lower cover plate containing an outlet. The upper cover plate, the intermediate functional plate, and the lower cover plate are irreversibly bonded together by thermal compression bonding.

The intermediate functional plate includes an upper continuous phase channel, an upper droplet shearing unit, and an upper droplet collecting channel, wherein the continuous phase enters through the continuous phase channel, and the dispersed phase enters through the dispersed phase channel, and is sheared by the continuous phase in the droplet shearing unit to form droplets, which are collected in the droplet collecting channel and flow out into the receiving phase through a lower cover plate containing an outlet; the lower cover plate includes a lower dispersed phase channel and an outlet.

The upper droplet shearing unit consists of a plurality of droplet micro-shearing units. Each droplet micro-shearing unit consists of two continuous phase channels, one dispersed phase channel, one necking shearing port, and one droplet channel. The plurality of droplet micro-shearing units described in the present disclosure are preferably 72 droplet micro-shearing units.

The present disclosure provides gelatin sponge microspheres, prepared by the method described in any one of the above technical solutions.

Compared with the prior art, the present disclosure provides a method for preparing gelatin sponge microspheres, including the following steps: A) dissolving gelatin in deionized water to obtain a dispersed phase solution; mixing vegetable oil and a surfactant to obtain a continuous phase solution; mixing vegetable oil and a surfactant to obtain a receiving phase solution; B) injecting the dispersed phase solution and the continuous phase solution respectively into a microfluidic device to obtain gelatin droplets; C) introducing the gelatin droplets into the receiving phase solution, cooling, and solidifying to obtain crude product of gelatin sponge microspheres; D) adding the crude product of gelatin sponge microspheres to the first crosslinking solution for preliminary crosslinking; after removing the upper layer solution, adding the second crosslinking solution for further crosslinking to obtain semi-finished gelatin sponge microspheres; E) removing the upper layer solution from the semi-finished gelatin sponge microspheres, and washing to obtain the gelatin sponge microsphere product. The present disclosure uses an alcohol-containing crosslinking solution for microsphere crosslinking. On the one hand, the miscibility of oil solution and alcohol can be utilized to remove oil solution from the surface of the microspheres, allowing the crosslinking agent to quickly contact the microspheres and undergo crosslinking. On the other hand, the osmotic pressure of the alcohol solution can be used to prevent the microspheres from swelling, which is beneficial for preparing small-sized droplets. Using an alcohol-containing crosslinking solution for microsphere crosslinking can maintain the droplet morphology, making the surface of the crosslinked microspheres smooth and wrinkle-free.

### BRIEF DESCRIPTION OF THE DRAWINGS

FIG. 1. Size and morphology of microspheres obtained at different isopropanol concentrations;

FIG. 2. Non-gradient crosslinking: the volume fraction of glutaraldehyde in crosslinking solution 2 is 0.1%;

FIG. 3. Non-gradient crosslinking: the volume fraction of glutaraldehyde in crosslinking solution 2 is 10%;

FIG. 4. Gradient crosslinking: the volume fraction of glutaraldehyde in crosslinking solutions 1 and 2 is 0.1%;

FIG. 5. Gradient crosslinking: the volume fractions of glutaraldehyde in crosslinking solutions 1 and 2 are 0.1% and 10%, respectively;

FIG. 6. Gradient crosslinking: the volume fractions of glutaraldehyde in crosslinking solutions 1 and 2 are 1% and 2%, respectively;

FIG. 7. Gradient crosslinking: the volume fractions of glutaraldehyde in crosslinking solutions 1 and 2 are 1% and 10%, respectively;

FIG. 8. Morphological images of microspheres after crosslinking in solutions with different organic solvents; (a) droplets, (b) isopropanol, (c) n-octanol, (d) n-butanol, (e) propylene glycol, (f) acetone;

FIG. 9. Model diagram of a multi-channel microfluidic chip;

FIG. 10. Composition of a microfluidic chip: (a) upper cover plate; (b) intermediate functional plate; (c) lower cover plate;

FIG. 11. Composition of the intermediate functional plate of the microfluidic chip: (A) upper droplet collecting channel; (B) upper continuous phase channel; (H) upper droplet shearing unit;

FIG. 12. Design diagram of droplet micro-shearing unit (partial view of H in FIG. 11): (a) continuous phase channel; (b) dispersed phase channel; (c) necking shearing port; (d) droplet channel;

FIG. 13. Composition of the lower cover plate of the microfluidic chip: (C) lower dispersed phase channel; (D) outlet;

FIG. 14. Droplets of different sizes: (a) 100 μm; (b) 200 μm; (c) 300 μm; (d) 400 μm; (e) 500μ; (f) 600μ; (g) 700μ; (h) 800 μm; (i) 900 μm; (j) 1000 μm;

FIG. 15. Morphology of microspheres obtained with different crosslinking solutions: (a) experimental group 1; (b) experimental group 2; (c) experimental group 3; (d) experimental group 4;

FIG. 16. Design diagram of droplet micro-shearing unit (non-necking structure);

FIG. 17. Photographs of gelatin droplets: (a) necking structure; (b) non-necking structure.

### DETAILED DESCRIPTION OF THE EMBODIMENTS

The present disclosure provides gelatin sponge microspheres and preparation method thereof. Those skilled in the art can refer to the content herein and appropriately improve the process parameters to achieve the desired result. It should be particularly noted that all similar substitutions and modifications are obvious to those skilled in the art and fall within the scope of protection of the present disclosure. The methods and applications of the present disclosure have been described through preferred embodiments. Those skilled in the art will be able to modify or appropriately change and combine the methods and applications described herein without departing from the spirit and scope of the present disclosure, in order to realize and apply the technology of the present disclosure.

The present disclosure provides a method for preparing gelatin sponge microspheres, including the following steps:
A) dissolving gelatin in deionized water to obtain a dispersed phase solution; mixing vegetable oil and a surfactant to obtain a continuous phase solution; mixing vegetable oil and a surfactant to obtain a receiving phase solution;
B) injecting the dispersed phase solution and the continuous phase solution respectively into a microfluidic device to obtain gelatin droplets;
C) introducing the gelatin droplets into the receiving phase solution, cooling, and solidifying to obtain crude product of gelatin sponge microspheres;
D) adding the crude product of gelatin sponge microspheres to the first crosslinking solution for preliminary crosslinking; after removing the upper layer solution, adding the second crosslinking solution for further crosslinking to obtain semi-finished gelatin sponge microspheres;
E) removing the upper layer solution from the semi-finished gelatin sponge microspheres, and washing to obtain the gelatin sponge microsphere product.

The method for preparing gelatin sponge microspheres provided by the present disclosure first prepares solutions:

Preparation of a dispersed phase solution: In an environment of 35-70 °C, gelatin is dissolved in deionized water, stirred to obtain a gelatin solution, and then the gelatin solution is maintained at 35-70 °C for subsequent use.

The gel strength of the gelatin of the present disclosure is 100-300 bloom, 100 bloom, 110 bloom, 120 bloom, 130 bloom, 140 bloom, 150 bloom, 160 bloom, 170 bloom, 180 bloom, 190 bloom, 200 bloom, 210 bloom, 220 bloom, 230 bloom, 240 bloom, 250 bloom, 260 bloom, 270 bloom, 280 bloom, 290 bloom, 300 bloom.

The mass fraction of gelatin in the dispersed phase solution is 1%-50%, in particular 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%; or any value within the range of the above two.

The stirring time is 0.5-2 h; in particular, it can be 0.5 h, 1 h, or 2 h.

Preparation of a continuous phase solution: at room temperature, a continuous phase solvent is used, to which a surfactant is added, and stirred to prepare the continuous phase solution.

In the continuous phase solution, the mass ratio of the vegetable oil to the surfactant is 100:(1-10); in particular, it can be 100:1, 100:2, 100:3, 100:4, 100:5, 100:6, 100:7, 100:8, 100:9 or 100:10.

In some specific embodiments, the vegetable oil includes one or more of soybean oil, peanut oil, and corn oil; the surfactant is one or more of Span 80, Span 85, and PGPR, and the surfactant mass fraction is 0.5%-10%; in particular, it can be 0.5%, 1%, 2%, 3%, 4%, 5%, 6%, 7%, 8%, 9%, 10%, or any value between the above two.

Preparation of a receiving phase solution: At room temperature, a continuous phase solvent is used, to which a surfactant is added, and stirred to prepare the receiving phase solution, and the receiving phase solution is placed in an environment of 0-8 °C.

In the receiving phase solution of the present disclosure, the mass ratio of the vegetable oil to the surfactant is 100:(0.1-2); in particular, it can be 100:0.1, 100:0.2, 100:0.3, 100:0.4, 100:0.5, 100:0.6, 100:0.7, 100:0.8, 100:0.9, 100:1.0, 100:1.1, 100:1.2, 100:1.3, 100:1.4, 100:1.5, 100:1.6, 100:1.7, 100:1.8, 100:1.9, 100:2.0; or any value between the above two.

The above-mentioned vegetable oils and surfactants have been clearly described and will not be repeated herein.

The dispersed phase solution and the continuous phase solution are injected respectively into the microfluidic device to obtain gelatin droplets.

In some specific embodiments, the pressure of the dispersed phase is 0.1 kPa to 500 kPa, in particular 1 kPa, 10 kPa, 50 kPa, 100 kPa, 150 kPa, 200 kPa, 250 kPa, 300 kPa, 350 kPa, 400 kPa, 450 kPa, 500 kPa. The pressure of the continuous phase is 0.05 MPa to 1 MPa. In particular, it can be 0.1 MPa, 0.2 MPa, 0.3 MPa, 0.4 MPa, 0.5 MPa, 0.6 MPa, 0.7 MPa, 0.8 MPa, 0.9 MPa, 1 MPa.

The microfluidic device of the present disclosure includes a constant temperature environment chamber, a microfluidic constant pressure pump, and a microfluidic chip, wherein the microfluidic chip is a multi-channel microfluidic chip with a flow focusing structure.

The present disclosure uses a multi-channel microfluidic chip, which can not only obtain gelatin microspheres with uniform size, but also increase the microsphere yield and reduce production costs.

The multi-channel microfluidic chip consists of an upper cover plate containing an inlet and an outlet, an intermediate functional plate containing microchannels, and a lower cover plate containing an outlet. The upper cover plate, the intermediate functional plate, and the lower cover plate are irreversibly bonded together by thermal compression bonding.

The intermediate functional plate includes an upper continuous phase channel, an upper droplet shearing unit, and an upper droplet collecting channel. The continuous phase enters through the continuous phase channel, and the dispersed phase enters through the dispersed phase channel. The dispersed phase is sheared by the continuous phase in the droplet shearing unit to form droplets. The droplets are collected in the droplet collecting channel and flow out into the receiving phase through a lower cover plate containing an outlet. The lower cover plate includes a lower dispersed phase channel and an outlet.

The upper droplet shearing unit consists of a plurality of droplet micro-shearing units, each consisting of two continuous phase channels, one dispersed phase channel, one necking shearing port, and one droplet channel.

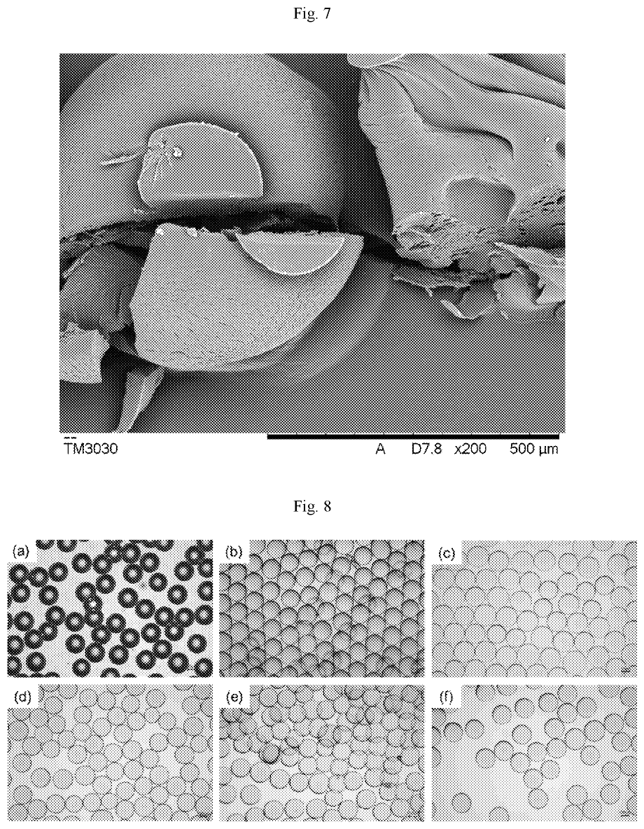

The inventors have discovered that if the droplet micro-shearing unit lacks a necking shearing port design, wall adhesion is likely to occur during droplet preparation, resulting in uneven droplet size (as shown in FIG. 7). The necking shearing port design of the droplet micro-shearing unit can effectively solve this problem and ensure the stability of droplet shearing (as shown in FIG. 8).

Gelatin droplets are introduced into the receiving phase solution, cooled, and solidified to obtain crude product of gelatin sponge microspheres.

In some specific embodiments, the cooling temperature in step C) is <30 °C; more preferably 0-8 °C.

The crude product of gelatin sponge microspheres are added to the first crosslinking solution for preliminary crosslinking. After removing the upper layer solution, a second crosslinking solution is added for further crosslinking to obtain semi-finished gelatin sponge microspheres.

According to the present disclosure, the first crosslinking solution includes water, a crosslinking agent, and an organic solvent.

According to the present disclosure, the crosslinking agent is an aldehyde organic compound; in particular, it can be formaldehyde, glutaraldehyde, or a glutaraldehyde solution containing 30% isopropanol;

The organic solvent is one of n-octanol, n-butanol, propylene glycol, isopropanol, or acetone; the volume fraction of the crosslinking agent in the first crosslinking solution is 0.1%-1%, in particular 0.1%, 0.2%, 0.3%, 0.4%, 0.5%, 0.6%, 0.7%, 0.8%, 0.9%, 1%; or any value within the range between any two of the foregoing values.

The volume concentration of the organic solvent is 25%-50%; in particular, it can be 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%; or any value within the range between any two of the foregoing values.

The time of the preliminary crosslinking described in the present disclosure is 30 min.

In some specific embodiments, the second crosslinking solution includes water, a crosslinking agent, and an organic solvent, wherein the crosslinking agent is an aldehyde organic compound; the organic solvent is one of n-octanol, n-butanol, propylene glycol, isopropanol, or acetone; the volume fraction of the crosslinking agent in the first crosslinking solution is 0.1%-10%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%; or any value within the range of the above two.

The organic solvent solution volume concentration is 25%-50%; the time of the further crosslinking is 30 min.

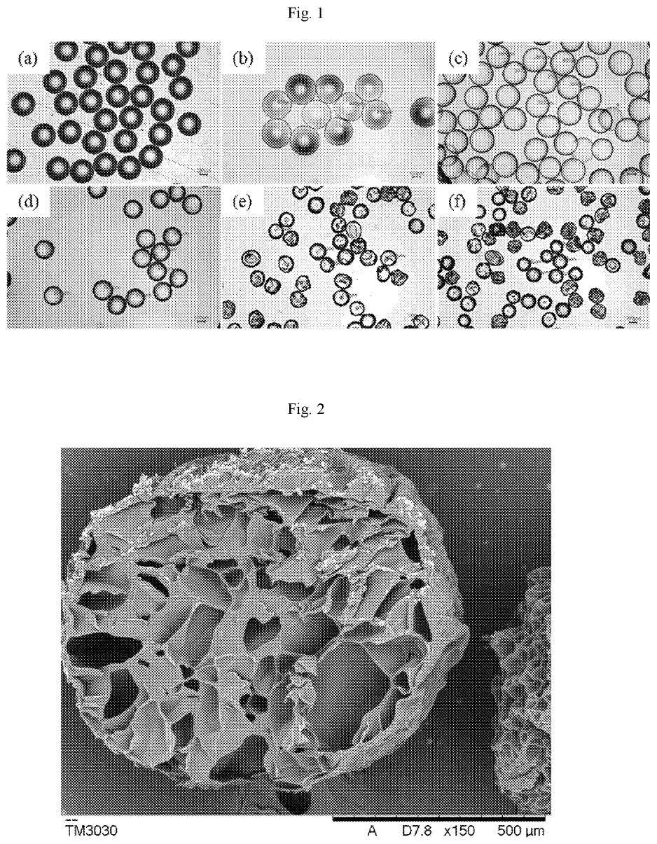

The present disclosure creatively adopts a gradient crosslinking method to avoid the formation of a dense shell layer on the surface of gelatin droplets when they come into contact with a high concentration of crosslinking agent, which would prevent the crosslinking agent from entering the droplet and causing inconsistent crosslinking levels inside and outside the microspheres, resulting in a core-shell structure formed under tensile stress (as shown in FIG. 1). The gradient crosslinking method allows the microspheres to be preliminarily crosslinked and fixed inside and outside under a low concentration of crosslinking agent, retaining the pores on the surface of the microspheres to facilitate the entry of the crosslinking agent, and then further crosslinked and fixed under a high concentration of crosslinking agent, thereby ensuring that the crosslinking level inside and outside the microspheres is consistent (as shown in FIG. 2).

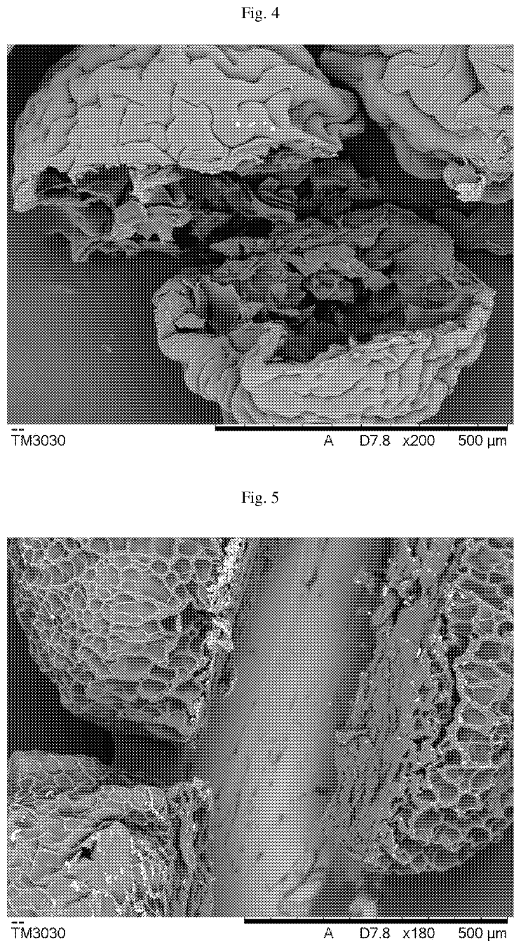

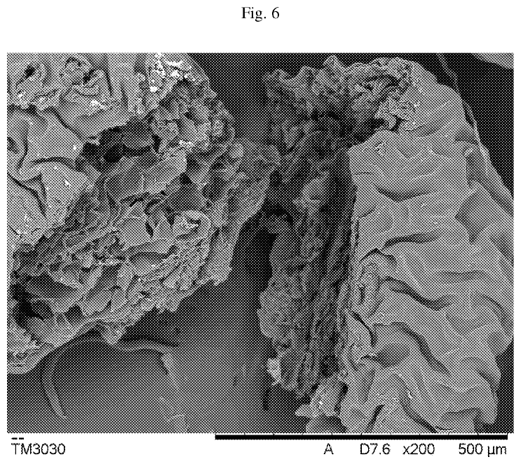

The present disclosure uses a crosslinking solution containing organic solvents for droplet crosslinking, which has three main advantages.
1) Vegetable oil adheres to the surface of the microspheres, preventing the crosslinking agent from contacting the microspheres, while the organic solvent can dissolve the vegetable oil on the surface of the microspheres, allowing the crosslinking agent to quickly contact and solidify with the microspheres, thus shortening the crosslinking time of the microspheres;
2) As the crosslinking agent and the oil solution are immiscible, the concentration of the crosslinking agent in actual contact with the microspheres is relatively high, which makes the surface of the microspheres crosslink rapidly to form a dense structure, resulting in uneven crosslinking degree between the inside and outside of the microspheres. However, by using a crosslinking solution containing an organic solvent for droplet crosslinking, the crosslinking agent can be uniformly mixed in the crosslinking solution, achieving a uniform crosslinking effect (as shown in FIG. 5).
3) A certain concentration of crosslinking solution containing an organic solvent can maintain the osmotic pressure inside and outside the microspheres, preventing the microspheres from shrinking and expanding, thus making the surface of the microspheres smooth and wrinkle-free, and facilitating the preparation of small-sized microspheres (for example, when preparing 100 μm gelatin microspheres, if the microspheres expand, the initial gelatin droplets need to be <100 μm, and the process will be more difficult for microspheres of even smaller sizes) (as shown in FIG. 6 and Table 1).

The upper layer solution is removed from the semi-finished gelatin sponge microspheres, which is then washed to obtain gelatin sponge microspheres.

The washing described in the present disclosure is washing with a cleaning agent followed by washing with deionized water; the cleaning agent is one of n-octanol, n-butanol, propylene glycol, isopropanol, or acetone; the volume concentration of the cleaning agent solution is not less than 75%.

In some embodiments, after removing the upper layer solution from the semi-finished gelatin sponge microspheres, a cleaning agent is added to wash the gelatin sponge microspheres until no continuous phase solvent residue remains; then, the microspheres are washed with deionized water to remove the residual cleaning agent, thus obtaining the gelatin sponge microspheres.

The present disclosure provides a gelatin sponge microsphere, prepared by any one of the methods described in the above technical solutions.

The present disclosure has already clearly described the above preparation method, which will not be repeated herein.

It should be understood that the expression “one or more of . . . ” refers individually to each of the objects described after the expression, as well as various different combinations of two or more of the described objects, unless otherwise understood from the context and usage. The expression “and/or” in conjunction with three or more described objects should be understood to have the same meaning, unless otherwise understood from the context.

The terms “including,” “having,” or “containing,” include the use of their grammatical synonyms, and should generally be understood as open-ended and non-limiting, e.g., not excluding other undescribed elements or steps, unless otherwise specifically stated or understood from the context.

In the present application, the term “and/or” describes the relationship between related objects, indicating that three relationships can exist. For example, A and/or B can represent: A exists alone, A and B exist simultaneously, or B exists alone. Among these, A, B can be singular or plural.

In the present application, “at least one” means one or more, and “multiple” means two or more. Expression “at least one of the following” or the like refers to any combination of them, including any combination of single items or multiple items.

It should be understood that the order of steps or the order in which certain actions are performed is not important as long as the present disclosure remains operable. Furthermore, two or more steps or actions may be performed simultaneously.

Any and all examples or exemplary language used herein, such as “e.g.” or “including,” are intended merely to better illustrate the present disclosure and do not constitute a limitation on the scope of the present disclosure. No language in this specification should be construed as indicating that any unclaimed element is essential to the practice of the present disclosure.

Furthermore, the numerical ranges and parameters used to define the present disclosure are approximate values, and the relevant values in the specific examples have been presented as precisely as possible herein. However, any numerical value inevitably contains a standard deviation due to the individual testing method. Therefore, unless otherwise specified, it should be understood that all ranges, quantities, values and percentages used in this disclosure are modified by “about”. Herein, “about” generally refers to an actual value plus or minus 10%, 5%, 1% or 0.5% of a specific value or range.

It should be understood that in the various examples of the present application, the sequence number of the above processes does not imply the order of implementation. Some or all steps can be implemented in parallel or sequentially. The implementation order of each process should be determined by its function and internal logic, and should not constitute any limitation on the implementation process of the examples of the present application.

The examples and comparative examples set forth herein provide certain implementations of the present disclosure. However, this does not mean that the effects of the present disclosure can only be achieved in these cases.

To further illustrate the present disclosure, the following describes in detail the gelatin sponge microspheres and preparation method thereof provided by the present disclosure in conjunction with the examples.

### Example 1

A method for preparing gelatin sponge microspheres, belonging to the technical field of biomedicine and polymer materials, included the following steps:
a. preparation of a dispersed phase solution: gelatin with a gel strength of 300 bloom was dissolved in deionized water in a 60 °C oil bath to prepare a gelatin solution with a mass fraction of 25%; then the gelatin solution was placed in a 60 °C oil bath environment and maintained for subsequent use;
b. preparation of a continuous phase solution: soybean oil was used, to which PGPR was added, and stirred at 300 rpm for 2 h to prepare a continuous phase solution with a PGPR mass fraction of 2%;
c. preparation of a receiving phase solution: soybean oil was used, to which PGPR was added, and stirred at 300 rpm for 2 h to prepare a receiving phase solution with a PGPR mass fraction of 0.5%; and the receiving phase solution was placed in an environment of 0-8 °C;
d. preparation of crosslinking solution 1: deionized water was used, to which isopropanol and glutaraldehyde were added, and stirred at 300 rpm for 2 h to prepare crosslinking solution 1 containing organic solvent with a glutaraldehyde volume fraction of 0.1%;
e. preparation of crosslinking solution 2: deionized water was used, to which isopropanol and glutaraldehyde were added, and stirred at 300 rpm for 2 h to prepare crosslinking solution 2 containing organic solvent with a glutaraldehyde volume fraction of 1% and an isopropanol volume concentration consistent with crosslinking solution 1;
f. the dispersed phase solution in step a and the continuous phase solution in step b were injected into a microfluidic device respectively, with a dispersed phase pressure of 0.06 MPa and a continuous phase pressure of 0.34 MPa, to obtain gelatin droplets of the expected size; in this step, the droplet size was achieved by controlling the dispersed phase pressure, continuous phase pressure, and microfluidic device size to reach the expected goal;
g. the gelatin droplets obtained in step f were cooled and solidified by the receiving phase solution in step c to obtain crude product of gelatin sponge microspheres;
h. the upper layer solution was removed from the crude product of gelatin sponge microspheres in step g; the crude product of gelatin sponge microspheres was subjected to gradient crosslinking, that was, the crosslinking solution 1 from step d was first added, crosslinked for 30 min to perform preliminary crosslinking; then the upper layer solution was removed, and the crosslinking solution 2 from step e was added, crosslinked for 30 min to perform further crosslinking, which yields the semi-finished gelatin sponge microspheres;
i. the upper layer solution was removed from the semi-finished gelatin sponge microspheres in step h; an isopropanol solution was added to wash the aforementioned crude product of gelatin sponge microspheres until there was no continuous phase solvent residue on the surface of the microspheres; then the microspheres was washed with deionized water 2-5 times to remove the isopropanol solution and other residues, which yields gelatin sponge microspheres that met the requirements.

The obtained gelatin sponge microspheres were then examined under a microscope to observe their shape characteristics. FIG. 1 shows the size and morphology of microspheres obtained under different isopropanol concentrations; (a) droplets, (b) 25% isopropanol, (c) 30% isopropanol, (d) 50% isopropanol, (e) 90% isopropanol, (f) 100% isopropanol. As can be seen from FIG. 1, under the same droplet conditions, as the isopropanol concentration increases, the size of the obtained microspheres gradually decreases, and the surface morphology and uniformity of the microspheres gradually deteriorate. When the concentration of isopropanol is <50%, the surface of the microspheres is relatively smooth; and when the isopropanol concentration is <25%, the microsphere shrinkage ratio is smaller and swelling occurs.

TABLE 1
Relationship between droplet and microsphere size

| | Droplet | Isopropanol | Micro- | Shrinkage ratio | |
|---|---|---|---|---|---|
| | size | volume | sphere | (droplet/micro- | |
| No. | [symbol] | [symbol] | size(μm) | sphere) | Note |
| 1 | 383 | 25 | 459 | 0.83 | FIG. 1(b) |
| 2 | | 30 | 385 | 0.99 | FIG. 1(c) |
| 3 | | 50 | 313 | 1.22 | FIG. 1(d) |
| 4 | | 90 | 262 | 1.46 | FIG. 1(e) |
| 5 | | 100 | 239 | 1.60 | FIG. 1(f) |

[symbol]

indicates data missing or illegible when filed

### Example 2

In this example, the dispersed phase solution, continuous phase solution, and receiving phase solution from Example 1 were used, and crosslinking solution 1 and crosslinking solution 2 were prepared with the isopropanol concentration of group 2. The method was as follows:

Preparation of crosslinking solution 1: deionized water was used, to which isopropanol and glutaraldehyde were added, and stirred at 300 rpm for 2 h, to prepare glutaraldehyde crosslinking solution 1 with an isopropanol volume concentration of 30%;

Preparation of crosslinking solution 2: deionized water was used, to which isopropanol and glutaraldehyde were added, and stirred at 300 rpm for 2 h, to prepare glutaraldehyde crosslinking solution 2 with an isopropanol volume concentration of 30%.

The remaining steps were the same as the preparation method in Example 1.

The obtained gelatin sponge microspheres were then examined under a scanning electron microscope to observe their shape characteristics.

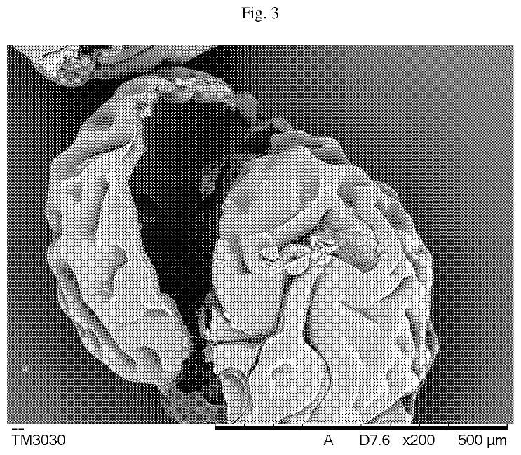

This example investigated the effects of non-gradient crosslinking and gradient crosslinking with different glutaraldehyde concentrations on the morphology of the microspheres; the morphology of the microspheres is shown in FIGS. 2-7. FIG. 2. Non-gradient crosslinking: the volume fraction of glutaraldehyde in crosslinking solution 2 is 0.1%; FIG. 3. Non-gradient crosslinking: the volume fraction of glutaraldehyde in crosslinking solution 2 is 10%; FIG. 4. Gradient crosslinking: the volume fraction of glutaraldehyde in crosslinking solutions 1 and 2 is 0.1%; FIG. 5. Gradient crosslinking: the volume fractions of glutaraldehyde in crosslinking solutions 1 and 2 are 0.1% and 10%, respectively; FIG. 6. Gradient crosslinking: the volume fractions of glutaraldehyde in crosslinking solutions 1 and 2 are 1% and 2%, respectively; FIG. 7. Gradient crosslinking: the volume fractions of glutaraldehyde in crosslinking solutions 1 and 2 are 1% and 10%, respectively.

As shown in FIGS. 2-7, when using non-gradient crosslinking, the microspheres exhibit a core-shell structure when the volume fraction of glutaraldehyde in crosslinking solution 2 is 10%. The high concentration of glutaraldehyde on the surface of the microspheres forms a dense shell with a high degree of crosslinking, which restricts the diffusion of the crosslinking agent, while the lower degree of crosslinking inside leads to shrinkage stress during subsequent processing, causing separation between the interior and the shell. Furthermore, when the volume fraction of glutaraldehyde in crosslinking solution 2 is 10%, the overall density of the microspheres increases.

TABLE 2
Parameter table for non-gradient crosslinking
and gradient crosslinking

| | Volume fraction of | Volume fraction of | |
|---|---|---|---|
| | glutaraldehyde in | glutaraldehyde in | |
| | crosslinking solution 1 | crosslinking solution | |
| No. | [symbol] | [symbol] | Note |
| 1 | 0 | 0.1 | Non-gradient |
| | | | crosslinking, see [symbol] |
| 2 | 0 | 10 | Non-gradient |
| | | | crosslinking, see [symbol] |
| 3 | 0.1 | 0.1 | Gradient cross- |
| | | | linking, see FIG. 4 |
| 4 | 0.1 | 10 | Gradient cross- |
| | | | linking, see FIG. 5 |
| 5 | 1 | 2 | Gradient cross- |
| | | | linking, see FIG. 6 |
| 6 | 1 | 10 | Gradient cross- |
| | | | linking, see FIG. 7 |

[symbol]

indicates data missing or illegible when filed

### Example 3

In this example, the dispersed phase solution, continuous phase solution, and receiving phase solution from Example 2 were used, and crosslinking solutions 1 and 2 containing organic solvents were prepared with a glutaraldehyde volume fraction of 0.5% and 2%, respectively. The method was as follows:

\textcircled{1} preparation of crosslinking solution 1: deionized water was used, to which n-octanol and glutaraldehyde were added, and stirred at 300 rpm for 2 h, to prepare crosslinking solution 1 with an n-octanol volume concentration of 30% and a glutaraldehyde volume fraction of 0.5%;

Preparation of crosslinking solution 2: deionized water was used, to which n-octanol and glutaraldehyde were added, and stirred at 300 rpm for 2 h, to prepare crosslinking solution 2 with an n-octanol volume concentration of 30% and a glutaraldehyde volume fraction of 2%.

\textcircled{2} Preparation of crosslinking solution 1: deionized water was used, to which n-butanol and glutaraldehyde were added, and stirred at 300 rpm for 2 h, to prepare crosslinking solution 1 with an n-butanol volume concentration of 30% and a glutaraldehyde volume fraction of 0.5%;

Preparation of crosslinking solution 2: deionized water was used, to which n-butanol and glutaraldehyde were added, and stirred at 300 rpm for 2 h, to prepare crosslinking solution 2 with an n-butanol volume concentration of 30% and a glutaraldehyde volume fraction of 2%.

\textcircled{3} Preparation of crosslinking solution 1: deionized water was used, to which propylene glycol and glutaraldehyde were added, and stirred at 300 rpm for 2 h, to prepare crosslinking solution 1 with a propylene glycol volume concentration of 30% and a glutaraldehyde volume fraction of 0.5%;

Preparation of crosslinking solution 2: deionized water was used, to which propylene glycol and glutaraldehyde were added, and stirred at 300 rpm for 2 h, to prepare crosslinking solution 2 with a propylene glycol volume concentration of 30% and a glutaraldehyde volume fraction of 2%.

\textcircled{4}Preparation of crosslinking solution 1: deionized water was used, to which acetone and glutaraldehyde were added, and stirred at 300 rpm for 2 h, to prepare crosslinking solution 1 with an acetone volume concentration of 30% and a glutaraldehyde volume fraction of 0.5%;

Preparation of crosslinking solution 2: deionized water was used, to which acetone and glutaraldehyde were added, and stirred at 300 rpm for 2 h, to prepare crosslinking solution 2 with an acetone volume concentration of 30% and a glutaraldehyde volume fraction of 2%.

The remaining steps were the same as the preparation method in Example 1.

The obtained gelatin sponge microspheres were then examined under a microscope to observe their shape characteristics.

This example investigated the effect of crosslinking solutions containing different organic solvents on the morphology of the microspheres. The morphology of the microspheres is shown in FIG. 8.

FIG. 8. Morphological images of microspheres after crosslinking in solutions with different organic solvents; (a) droplets, (b) isopropanol, (c) n-octanol, (d) n-butanol, (e) propylene glycol, (f) acetone.

As shown in FIG. 8, there was no significant difference in the morphology and shrinkage rate of microspheres after crosslinking droplets with crosslinking solutions prepared with different organic solvents.

TABLE 3
Statistical table of the influence of crosslinking solutions containing
different organic solvents on microsphere morphology

| | Droplet | Type of | Micro- | Shrinkage ratio | |
|---|---|---|---|---|---|
| | size | organic | sphere | (droplet/micro- | |
| No. | [symbol] | [symbol] | size (μm) | sphere) | Note |
| 1 | 329 | Isopropanol | 314 | 1.05 | FIG. 8(b) |
| 2 | | N-octanol | 341 | 0.96 | FIG. 8(c) |
| 3 | | N-butanol | 314 | 1.05 | FIG. 8(d) |
| 4 | | Propylene | 318 | 1.03 | FIG. 8(e) |
| 5 | | Acetone | 329 | 1.00 | FIG. 8(f) |

[symbol]

indicates data missing or illegible when filed

### Example 4

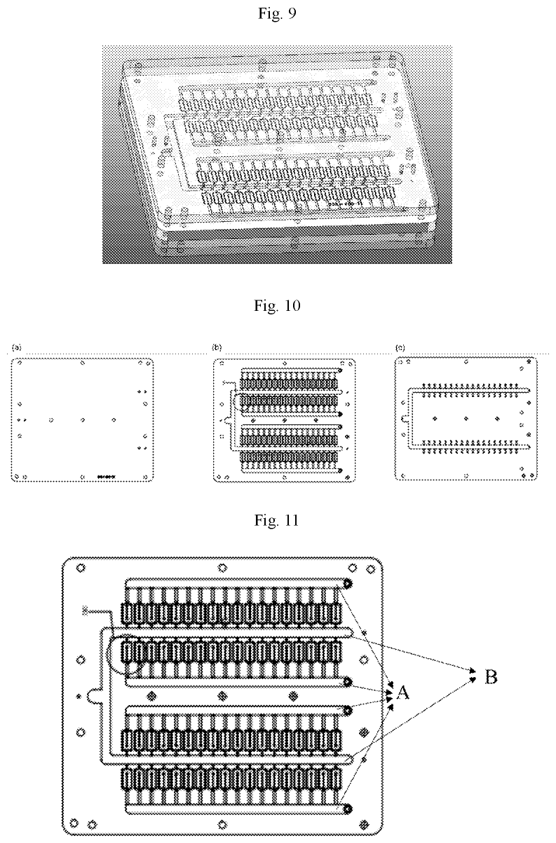

In this example, the dispersed phase solution, continuous phase solution, and receiving phase solution from Example 2 were used, and gelatin droplets of different sizes were prepared using different microfluidic chips. The microfluidic chip design diagrams are shown in FIGS. 9-13.

Each microfluidic chip consists of an upper cover plate containing an inlet and an outlet, an intermediate functional plate containing microchannels, and a lower cover plate containing an outlet. The upper cover plate, the intermediate functional plate, and the lower cover plate are irreversibly bonded together by thermal compression bonding.

The intermediate functional plate of different microfluidic chips includes an upper continuous phase channel, an upper droplet shearing unit, and an upper droplet collecting channel. The continuous phase enters through the continuous phase channel, and the dispersed phase enters through the dispersed phase channel. The dispersed phase is sheared by the continuous phase in the droplet shearing unit to form droplets. The droplets are collected in the droplet collecting channel and flow out into the receiving phase through the lower cover plate containing the outlet. The lower cover plate includes a lower dispersed phase channel and an outlet.

The upper droplet shearing unit of different microfluidic chips consists of multiple droplet micro-shearing units. The droplet micro-shearing unit consists of two continuous phase channels, one dispersed phase channel, one necking shearing port, and one droplet channel.

The gelatin droplet preparation steps in this example are the same as those in Example 1.

The obtained gelatin droplets were then observed and measured under a microscope, and the size distribution of the gelatin droplets was statistically analyzed.

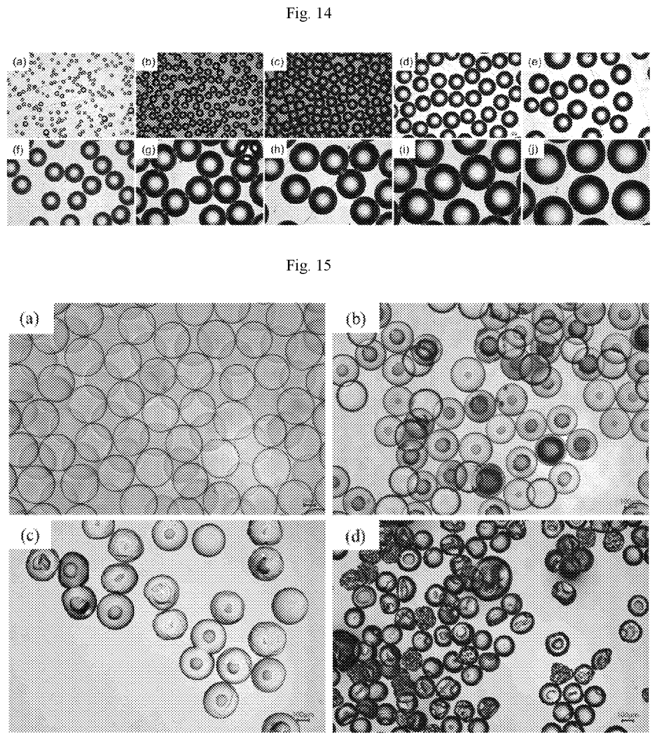

This example examined the preparation of gelatin droplets of different sizes by controlling the dispersed phase pressure, continuous phase pressure, and microfluidic device size. The morphology of the gelatin droplets is shown in FIG. 14.

As can be seen from FIG. 14, droplets of different sizes can be prepared by using different chip structure designs and different dispersed phase pressures and continuous phase pressures.

TABLE 4
Statistical table of gelatin droplets prepared with different dispersed
phase pressures, continuous phase pressures, and microfluidic chip sizes

| Chip | Chip size (μm) | | | | Internal | External | Droplet | CV | |
|---|---|---|---|---|---|---|---|---|---|
| model | a | b | c | d | Pressure | pressure | size(μm) | value | Note |
| 100*200 | 100 | 100 | 200 | 400 | 0.010 | 0.40 | 90-105 | 8% | FIG. 14 (a) |
| | | | | | 0.010 | 0.06 | 200-220 | 5% | FIG. 14 (b) |
| 150*250 | 150 | 150 | 250 | 400 | 0.004 | 0.05 | 320-330 | 5% | / |
| 200*300 | 200 | 200 | 300 | 500 | 0.012 | 0.48 | 200-220 | 6% | / |
| | | | | | 0.024 | 0.22 | 290-310 | 4% | FIG. 14 (c) |
| | | | | | 0.025 | 0.08 | 400-420 | 5% | FIG. 14 (d) |
| 350*750 | 350 | 350 | 750 | 900 | 0.014 | 0.07 | 450-470 | 4% | / |
| | | | | | 0.036 | 0.05 | 500-520 | 4% | FIG. 14 (e) |
| | | | | | 0.042 | 0.05 | 580-600 | 6% | FIG. 14 (f) |
| 400*800 | 400 | 400 | 800 | 1000 | 0.045 | 0.50 | 690-715 | 5% | FIG. 14 (g) |
| | | | | | 0.065 | 0.60 | 780-810 | 3% | FIG. 14 (h) |
| 500*900 | 500 | 500 | 900 | 1200 | 0.110 | 0.15 | 800-840 | 4% | / |
| | | | | | 0.110 | 0.20 | 900-930 | 5% | FIG. 14 (i) |
| | | | | | 0.060 | 0.34 | 1020~1070 | 7% | FIG. 14 (j) |

Note
CVvalue = standard deviation/average value*100%

### Example 5

This example investigated the effects of crosslinking with a crosslinking solution containing organic solvent and crosslinking with a crosslinking solution without organic solvent on the morphology of microspheres. The morphology of the microspheres is shown in FIG. 15.

In this example, experimental group 1, which used a crosslinking solution containing organic solvent, performed crosslinking using the dispersed phase solution, continuous phase solution, and receiving phase solution from Example 1 with crosslinking solution 1 containing glutaraldehyde in a volume fraction of 1% and isopropanol in a volume fraction of 30% for 30 min; and the remaining steps were the same as the preparation method in Example 1.

In this example, crosslinking experimental group 2, which used a crosslinking liquid without organic solvent, performed crosslinking on droplets using the dispersed phase solution, continuous phase solution, and receiving phase solution from Example 1 with a soybean oil solution containing glutaraldehyde in a volume fraction of 1% for 30 min; and the remaining steps were the same as the preparation method in Example 1.

In this example, experimental group 3, which used a crosslinking solution without organic solvent, performed crosslinking using the dispersed phase solution, continuous phase solution, and receiving phase solution from Example 1 with a crosslinking solution containing glutaraldehyde in a volume fraction of 1% for 30 min; and the remaining steps were the same as the preparation method in Example 1.

In this example, experiment group 4, which used a crosslinking solution without organic solvent, employed the dispersed phase solution, continuous phase solution, and receiving phase solution from Example 1; after the gelatin droplets were cooled and solidified by the receiving phase solution, they were washed with isopropanol and then crosslinked with a crosslinking solution containing glutaraldehyde in a volume fraction of 1%. The method is as follows:
(1) Gelatin droplets were obtained using the same preparation method as described in Example 1; after the obtained gelatin droplets were cooled and solidified by the receiving phase solution, crude product of gelatin sponge microspheres were obtained;
(2) After removing the upper layer solution from the crude product of gelatin sponge microspheres in step (1), isopropanol solution was added to wash the crude product of gelatin sponge microspheres until there was no continuous solvent residue on the surface of the microspheres, which were then crosslinked with a crosslinking solution containing glutaraldehyde in a volume fraction of 1% for 30 min to obtain semi-finished gelatin sponge microspheres.

Then the obtained gelatin sponge microspheres were examined under a microscope to check the shape characteristics of the microspheres.

FIG. 15. Morphology of microspheres obtained with different crosslinking solutions: (a) experimental group 1; (b) experimental group 2; (c) experimental group 3; (d) experimental group 4. As can be seen from FIG. 15, for microspheres obtained by crosslinking with the crosslinking solution containing organic solvent, the surface morphology is relatively smooth and the degree of crosslinking is uniform. For microspheres obtained by crosslinking with the crosslinking solution without organic solvent in experimental groups 2 and 3, the degree of crosslinking is not uniform, and the degree of crosslinking inside the microsphere is inconsistent with that on the surface of the microsphere. For microspheres obtained by crosslinking with the crosslinking solution without organic solvent in experimental group 4, most of them exhibited shrinking, which occurred after the microspheres were washed with isopropanol and the microspheres could not be completely restored during the crosslinking process.

### Example 6

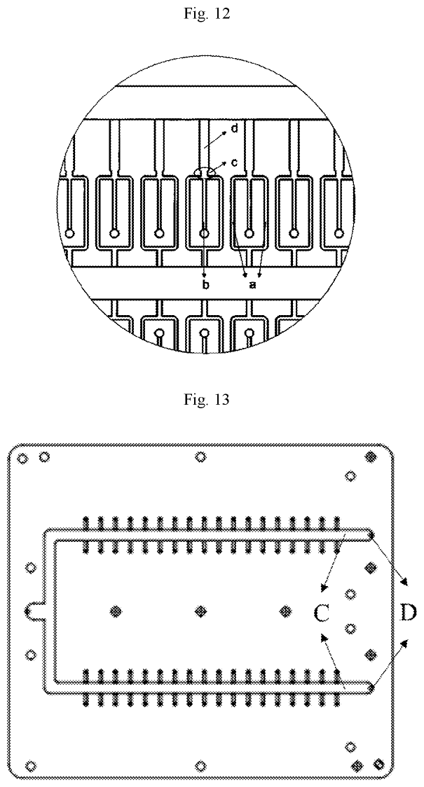

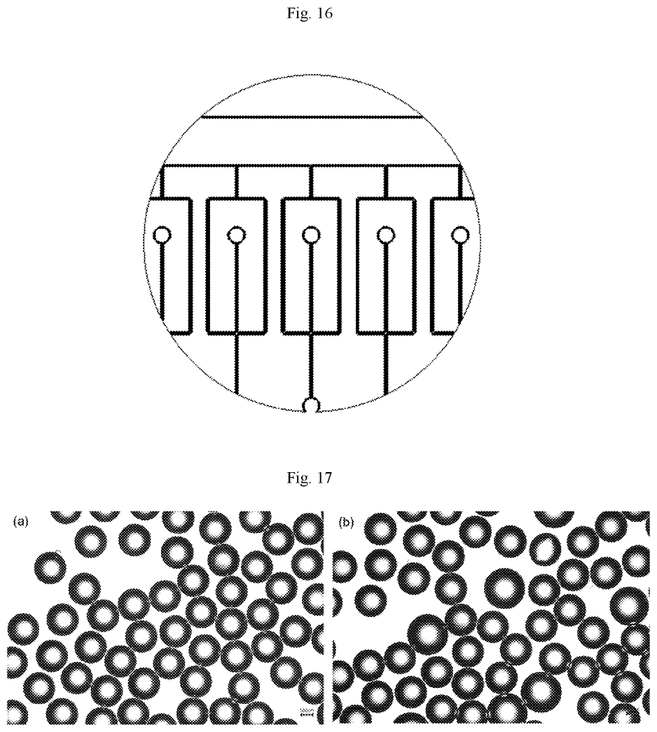

This example investigated the uniformity of droplets prepared using a droplet micro-shearing unit with a necking structure design (see FIG. 12) and without a necking structure design (see FIG. 16); the results are shown in FIG. 17.

The remaining steps were the same as the preparation method in Example 1.

The obtained gelatin droplets were observed under a microscope.

As can be seen from FIG. 17, the uniformity of gelatin droplets prepared by the non-necking structure was lower than that prepared by the necking structure. That was because the shearing force of the shearing unit of the non-necking structure chip was weaker, and the shearing stability was lower than that of the necking structure chip.

The above description is only a preferred embodiment of the present disclosure. It should be noted that for those skilled in the art, several improvements and modifications can be made without departing from the principle of the present disclosure, and these improvements and modifications should also be considered within the scope of protection of the present disclosure.

What is claimed is:

1. A method for preparing a gelatin sponge microsphere product, wherein the method comprises the following steps:
A) dissolving gelatin in deionized water to obtain a dispersed phase solution; mixing vegetable oil and a surfactant to obtain a continuous phase solution; mixing vegetable oil and a surfactant to obtain a receiving phase solution;
B) injecting the dispersed phase solution and the continuous phase solution respectively into a microfluidic device to obtain gelatin droplets;
C) introducing the gelatin droplets into the receiving phase solution, cooling, and solidifying to obtain crude product of gelatin sponge microspheres;
D) adding the crude product of gelatin sponge microspheres to the first crosslinking solution for preliminary crosslinking; after removing the upper layer solution, adding the second crosslinking solution for further crosslinking to obtain semi-finished gelatin sponge microspheres; the first crosslinking solution includes water, a crosslinking agent, and an organic solvent; the organic solvent is one of n-octanol, n-butanol, propylene glycol, isopropanol, or acetone;
E) removing the upper layer solution from the semi-finished gelatin sponge microspheres, and washing to obtain the gelatin sponge microsphere product.

2. The method according to claim 1, wherein the temperature for dissolving gelatin in step A) is 35-70 °C; the gel strength of gelatin is 100-300 bloom; the mass fraction of gelatin in the dispersed phase solution is 1%-50%; the stirring time is 0.5-2 h.

3. The method according to claim 1, wherein the vegetable oil in step A) comprises one or more of soybean oil, peanut oil, and corn oil; the surfactant is one or more of Span 80, Span 85, and PGPR; the mass fraction of the surfactant is 0.5%-10%;
in the continuous phase solution, the mass ratio of the vegetable oil to the surfactant is 100:(1:-10);
in the receiving phase solution, the mass ratio of the vegetable oil to the surfactant is 100:(0.1-2).

4. The method according to claim 1, wherein the pressure of the dispersed phase solution in step B) is 0.1 kPa-500 kPa, and the pressure of the continuous phase solution is 0.05 MPa-1 MPa.

5. The method according to claim 1, wherein the crosslinking agent in step D) is an aldehyde organic compound; the volume fraction of the crosslinking agent in the first crosslinking solution is 0.1%-1%, and the volume concentration of the organic solvent is 25%-50%; the time of the preliminary crosslinking is 10 min-60 min.

6. The method according to claim 1, wherein in step D), the second crosslinking solution includes water, a crosslinking agent and an organic solvent, wherein the crosslinking agent is an aldehyde organic compound; the organic solvent is one of n-octanol, n-butanol, propylene glycol, isopropanol or acetone; the volume fraction of the crosslinking agent in the second crosslinking solution is 0.1%-10%; the volume concentration of the organic solvent is 25%-50%; the time of the further crosslinking is 10 min-2 h.

7. The method according to claim 1, wherein the cooling temperature in step C) is 0−8 °C.

8. The method according to claim 1, wherein the washing in step E) is washing with a cleaning agent followed by washing with deionized water; the cleaning agent is one of n-octanol, n-butanol, propylene glycol, isopropanol, or acetone; the volume concentration of the cleaning agent solution is not less than 75%.

9. The method according to claim 1, wherein the microfluidic device in step B) comprises a constant temperature environment chamber, a microfluidic constant pressure pump, and a microfluidic chip, wherein the microfluidic chip is a multi-channel microfluidic chip with a flow focusing structure;
the multi-channel microfluidic chip consists of an upper cover plate containing an inlet and an outlet, an intermediate functional plate containing microchannels, and a lower cover plate containing an outlet; the upper cover plate, the intermediate functional plate, and the lower cover plate are irreversibly bonded together by thermal compression bonding;
the intermediate functional plate comprises an upper continuous phase channel, an upper droplet shearing unit, and a lower droplet collecting channel, wherein the continuous phase enters through the continuous phase channel, and the dispersed phase enters through the dispersed phase channel, and is sheared by the continuous phase in the droplet shearing unit to form droplets, which are collected in the droplet collecting channel and flow out into the receiving phase through a lower cover plate containing an outlet;
the upper droplet shearing unit consists of a plurality of droplet micro-shearing units, each droplet micro-shearing unit consists of two continuous phase channels, one dispersed phase channel, one necking shearing port, and one droplet channel.

10. A gelatin sponge microsphere product, wherein it is prepared by the method according claim 1.
